(19) United States
(12) Patent Application Publication
(10) Pub. No.: US 2002/0155502 A1
(43) Pub. Date: Oct. 24, 2002

(54) Affinity maturation by competitive selection

(75) Inventors: Robert F. Balint, Palo Alto, CA (US); Jeng-Horng Her, San Jose, CA (US); James W. Larrick, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Horizon Biotechnologies, Inc., Mountain View, CA

(21) Appl. No.: 09/999,413

(22) Filed: Oct. 30, 2001

Related U.S. Application Data
(60) Provisional application No. 60/245,039, filed on Oct. 30, 2000.

(51) Int. Cl. G01N 33/53; G01N 33/567; G01N 33/554; G01N 33/569

(52) U.S. Cl. 435/7.1; 435/7.2; 435/7.32

(57) ABSTRACT

The present invention provides a method of selecting binding pair members with enhanced binding affinity for the cognate binding partner relative to a reference binding pair member. In particular, the invention provides methods of selecting antibodies with enhanced affinity for an antigen relative to a reference antibody. This process, “affinity maturation”, thereby provides antibodies with superior binding capabilities.

### CROSS-REFERENCES TO RELATED APPLICATIONS

This application claims the benefit of priority of U.S. Provisional Application No. 60/245,039, filed Oct. 30, 2000, which is herein incorporated by reference.

### FIELD OF THE INVENTION

The present invention provides a method of selecting a binding pair member with enhanced binding affinity for a cognate binding partner relative to a reference binding pair member. In particular, the invention provides methods of selecting antibodies with enhanced affinity for an antigen relative to a reference antibody. This process, “affinity maturation”, thereby provides binding pair members, e.g., antibodies, with superior binding capabilities.

### BACKGROUND OF THE INVENTION

Reporter systems have been developed in which complementing fragments of a reporter molecule are each joined to a member of a binding pair. When the binding pair interacts, i.e., binds to one another, the complementing fragments are brought into proximity such that reporter activity is reconstituted (see, e.g., WO 00/71702). Other reporter systems can also be engineered in which reporter activation (or inhibition) is dependent on a binding interaction between a binding pair member linked to a member of a reporter system and its cognate binding partner, which can be linked to a subunit or inhibitor of the reporter. Such systems are useful for many applications, for example, the identification of analytes in a sample, tissue-localized activation of therapeutic and imaging reagents, as sensors in high-throughput screening of agonists/antagonists, high-throughput mapping of pair-wise protein-protein interactions, rapid selection of antibody fragments or other binding proteins that binding specifically to polypeptides of interest, rapid antigen identification for anti-cell and anti-tissue antibodies, rapid epitope identification for antibodies, and in cell-based screens for high-throughput selection of inhibitors of protein-protein interactions. In some of these applications, for example, the identification of antibodies or other binding proteins that specifically bind to a polypeptide of interest, it is desirable to select high-affinity binders. This basic system is limited, however, in its ability to discriminate on the basis of the affinity of interactors.

Antigen-specific antibodies can be produced by a variety of methods including hybridoma technology (e.g., Kohler & Milstein, Nature 256:495-497, 1975) or selection in vitro using phage or yeast display libraries (e.g., Hoogenboom et al., Immunotechnology 4:1-20, 1998; Boder & Wittrup, Methods Enzymol 328:430-44, 2000). However, antibodies derived from these methods often have sub-optimal binding affinities. Affinity discrimination among conspecific antibodies in vitro may be accomplished by equilibrating a mutagenic library of the antibody in question with soluble cognate antigen under conditions in which the concentrations of both antibody and antigen are maintained below the target equilibrium dissociation constant. Competition must be avoided to prevent abundant low-affinity variants from excluding rarer high-affinity variants. Operationally, there are two additional requirements: (1) the antibody library must be displayed by a vehicle such as a bacteriophage or a cell, which couples the antibody to its coding sequence, and (2) the antigen must be coupled to a tag which allows quantitative separation of antigen-bound antibody from unbound antibody. The main drawback of such procedures is that for most applications, affinities in the nanomolar K_{d} range are desired and these antibodies are sufficiently rare to be easily lost at sub-nanomolar working concentrations.

Such difficulties have led to selecting for lower dissociation rate constants (k_{d}, off-rates) to improve antibody affinities. Selection for lower off-rates is usually performed under saturating conditions where antigen-antibody complexes remaining intact after a time proportional to the inverse of the target off-rate can be recovered separately from dissociated antibodies. However, in these procedures it is frequently desired that off-rates for antibodies are in the range of 1×10⁻⁴ sec⁻¹, which corresponds to a half-life of ˜2 hours. Many antibodies and especially antigens undergo significant irreversible denaturation in vitro on such time scales at ambient or physiological temperatures. Also, off-rate selection in the absence of on-rate selection (lower kₐ) tends to bias the selection toward variants that refold into stable complexes and therefore tend to disfavor increased on-rates.

For most antibody applications, a successful protocol for affinity maturation in vitro will be one that produces improvements in both on-rate and off-rate while maintaining or increasing the specificity for the intended antigen. The current invention provides such a system. The invention provides methods and systems for the identification of test binding pair members that have a higher affinity than a reference binding pair member, i.e., an affinity matured or improved binding pair member. In one embodiment, the invention provides a method comprising a fragment complementation system that uses binding affinity as a selective criterion. The same principle of cell-based competitive selection of higher affinity variants may be used with any reporter system, which confers a selectable phenotype on the cells, such as color, or the ability to grow under restrictive conditions, and whose activation or inhibition can be made to depend on the interaction of two binding pair members.

### BRIEF SUMMARY OF THE INVENTION

In general, the method comprises introducing into a population of bacterial cells expression vectors comprising nucleic acid sequences encoding (a) a library of test binding pair members, (b) a cognate binding partner, (c) a competitor that has the properties of: i) competing with the reference binding pair member for binding to the cognate binding partner and ii) having an affinity for the cognate binding partner at least equal to that of the reference binding pair member. The vectors also encode a reporter system which is comprised of one, two, or three molecules, at least one of which is linked to (a) the test binding pair members, (b) the cognate binding partner, or (c) the competitor. The bacterial cells are then cultured under conditions wherein the reporter becomes active, conferring a selectable phenotype on the cells, when a test binding pair member binds to the cognate binding partner with a higher affinity than that of the reference binding pair member. Such higher affinity test binding pair members are identified by the phenotype of the cells relative to that conferred by the reference binding pair member.

In one embodiment, the method comprises introducing an expression vector comprising nucleic acid sequences encoding a library of test binding pair members linked to a fragment A of a marker into a population of bacterial cells, introducing into the population of cells an expression vector comprising nucleic acid sequences encoding the cognate binding partner linked to a fragment B of a marker (wherein the marker is active when the fragment A and the fragment B are in proximity); culturing the population of cells under conditions in which the library of test binding pair members linked to fragment A and the cognate binding partner linked to fragment B are expressed in the presence of a competitor that has the properties of: i) competing with the reference binding pair member for binding to the cognate binding partner and ii) having an affinity for the cognate binding partner at least equal to that of the reference binding pair member; wherein a test binding pair member having a higher affinity than the competitor binds to the cognate binding partner linked to fragment A; and selecting a cell in which the marker is active. The binding domain of the test binding pair member is typically at least 90% identical to the binding domain of the reference binding pair member. In many embodiments, the selecting step comprises selecting a cell in which the marker is more active than a reference standard of activity.

The competitor is often the reference binding pair member, but can also be an analog that binds similarly, i.e., with a comparable affinity, to the cognate binding partner. Frequently, the reference binding pair member is an antibody, in particular, a single chain antibody. In such embodiments, the test binding pair members linked to fragment A are typically single chain antibodies.

The reference binding pair member can also be a peptide or binding domain other than an antibody. For example, a reference binding pair member can be a peptide agonist or antagonist of a receptor.

In other embodiments, the cognate binding partner linked to fragment B is expressed at a concentration that is limiting, the competitor is expressed in an amount that is in excess over a concentration equivalent to its K_{d} for binding to the cognate binding partner, and the concentration of the test binding pair member linked to fragment A expressed in the cell population is substantially the same as that of the cognate binding partner linked to fragment B. Frequently, the concentration of the cognate binding partner linked to fragment B is one-tenth or less the concentration of the competitor and the competitor is in about 10-fold excess over a concentration equivalent to its K_{d} for binding to the cognate binding partner.

In some embodiments, the competitor is expressed from an expression vector comprising nucleic acid sequences encoding the competitor that is introduced into the cell population. Often, the competitor and the cognate binding partner linked to fragment B of the marker are encoded on one expression vector and can be expressed as a dicistronic transcript from a single promoter such as a trp-lac promoter.

In practicing the methods of the invention, the population of bacterial cells are often gram negative bacteria and the marker comprises a signal peptide.

In another aspect, the invention provides a bacterial cell comprising an expression vector comprising nucleic acid sequences encoding a member of a library of test binding pair members linked to a fragment A of a marker; an expression vector comprising nucleic acid sequences encoding a cognate binding partner linked to a fragment B of a marker; wherein the marker is active when the fragment A and the fragment B are in proximity; and a competitor that competes with a reference binding pair member for binding to the cognate binding partner and has an affinity for the cognate binding partner at least equal to that of the reference binding pair member. Often, the competitor is the reference binding pair member.

In some embodiments of the bacterial cell, the reference binding pair member is an antibody, in particular, a single chain antibody. The test binding pair members linked to fragment A can also be single chain antibodies.

Additionally, the cognate binding partner linked to fragment B can be expressed in the bacterial cell at a concentration that is limiting, the competitor can be expressed in an amount that is in excess over its K_{d} for binding to the cognate binding partner, and the concentration of the test binding pair member linked to fragment A expressed in the cell population can be substantially the same as that of the cognate binding partner linked to fragment B. Often, the concentration of the cognate binding partner linked to fragment B is one-tenth or less the concentration of the competitor. The competitor can also be in about 10-fold excess over its K_{d} for binding to the cognate binding partner.

In some embodiments of the bacterial cell, the competitor is expressed from an expression vector comprising nucleic acid sequences encoding the competitor. Often, the competitor and the cognate binding partner linked to fragment B of the marker are encoded on one expression vector and the competitor and cognate binding partner linked to fragment B are expressed as a dicistronic transcript from a single promoter such as a trp-lac promoter.

Furtheremore, the bacterial cell can be a gram negative bacterial cell and the marker can comprise a signal peptide.

The invention can be used with any reporter system that confers a selectable phenotype on the cells, e.g., color, the ability to grow in the presence of certain antibiotics, or the ability to utilize certain nutrient precursors for growth. In these systems, reporter signal generation is made to depend on the interaction of heterologous binding pair members. For example, multimeric reporters can be used in which one or more subunits are linked to binding pair members and/or the competitor in such a way that the binding of the competitor to the cognate binding pair member leads to inactivation of the reporter. Test binding pair members are identified by their ability to compete for binding to the cognate binding pair member, thereby activating the reporter.

Cell-based systems for affinity maturation have a significant advantage over in-vitro systems such as bacteriophage display for such applications. In in-vitro systems, populations of test binding pair members, displayed on phage, for example, may compete in solution for binding to a limiting amount of immobilized cognate binding partner. Those test binding pair members which bind to the cognate binding partner may be recovered by facile physical separation from those which do not. However, competition by affinity alone is not possible in vitro because the overwhelming abundance of low-affinity variants in the population will necessarily saturate the limiting cognate binding partner, and thereby prevent the necessarily rare higher-affinity variants in the population from being selected. Only cell-based systems can allow true competition by affinity alone because they do not allow test binding pair members to compete with each other, only with the competitor. The cells expressing each test binding pair member do not compete with each other or with the other cells for selection. They are selected solely on the basis of the strength of their phenotype, whether viability or color, etc. Since each test binding pair member has the same abundance inside the cell, the strength of its phenotype, i.e., its reporter activity, cannot depend on its abundance, but only on its affinity. Thus, in a cell-based system test binding pair members cannot be selected on the basis of abundance, but only on the basis of affinity.

### BRIEF DESCRIPTION OF THE DRAWINGS

FIG. 1 depicts expression constructs for competitive affinity maturation of an scFv.

FIG. 2 illustrates affinity maturation of an scFv by competitive selection. A “low affinity” scFv selected from a repertoire library or random CDR library is first co-expressed as a free “competitor” with the β-lactamase fragments fused to the antigen and the same scFV. This allows determination of the antibiotic concentration needed to prevent growth of cells expressing unimproved scFvs. The scFv fused to the ω198 fragment is mutagenized and selected for growth on the restrictive antibiotic concentration.

FIG. 3 depicts expression constructs for a single-chain Fv antibody fragment (scFv) selection from repertoire libraries or random CDR libraries. The scFv library is encoded on a phagemid plasmid for expression as the C-terminal fusion to the β-lactamase ω fragment via flexible linker ((G₄S)₃). The phage origin of replication (f1 ori) allow the scFv libraries to be archived as bacteriophage stocks, which can be used to quantitatively infect (high multiplicity of infection (m.o.i.)) cells expressing the antigen fused via a (G₄S)₃ linker to either end of the β-lactamase α fragment. pUC ori, p15A ori, origins of replication of compatible plasmids; lac prom, lac operon transcription promoter; trc prom, trp-lac fusion promoter; SP, signal peptide for secretion into the periplasm; cat, chloramphenicol resistance gene; kan, kanamycin resistance gene.

FIG. 4 shows a representative experiment in which scFv specific for the extra-cellular domain of human CD40 (CD40ED) were selected from a library of about 198 independent clones expressing human scFv based on the DP47 germline VH gene and the DPL3 germline V1 gene with VH CDR3s containing 12-16 amino acids of random sequence. Double break-point fusion means the antigen was fused to the C-terminus of the α197 fragment; double N-terminal fusion means the antigen was fused to the N-terminus of the sα197 fragment.

FIG. 5 illustrates anti-CD40 Trxpep competition. FIG. 5A shows expression constructs for determination of complementation groups among CD40-binding trxpeps by competition. FIG. 5B represents a tabulation of competitive relationships among 7 CD40-binding trxpeps.

Definitions

A “binding pair member” refers to a molecule that participates in a specific binding interaction with a binding partner, which can also be referred to as a “second binding pair member” or “cognate binding partner”. Binding pairs include antibodies/antigens, receptor/ligands, biotin/avidin, and interacting protein domains such as leucine zippers and the like. A binding pair member as used herein can be a binding domain, i.e., a subsequence of a protein that binds specifically to a binding partner.

The term “interaction” or “interacts” when referring to the interaction of binding pair members refers to specific binding to one another.

A “reference binding pair member” is a known binding pair member for which the practitioner wants to obtain a higher affinity binding analog i.e., an “improved” binding pair member.

An “affinity matured” or “improved” binding pair member is one that binds to the same site as an initial reference binding pair member, but has a higher affinity for that site.

Binding affinity is generally expressed in terms of equilibrium association or dissociation constants (Kₐ or K_{d}, respectively), which are in turn reciprocal ratios of dissociation and association rate constants (k_{d} and kₐ, respectively). Thus, equivalent affinities may correspond to different rate constants, so long as the ratio of the rate constants remains the same.

“Domain” refers to a unit of a protein or protein complex, comprising a polypeptide subsequence, a complete polypeptide sequence, or a plurality of polypeptide sequences where that unit has a defined function. The function is understood to be broadly defined and can be binding to a binding partner, catalytic activity or can have a stabilizing effect on the structure of the protein. “Domain” also refers to a structural unit of a protein or protein complex, comprising one or more polypeptide sequences where that unit has a defined structure which is recognizable within the larger structure of the native protein. The domain structure is understood to be semi-autonomous in that it may be capable of forming autonomously and remaining stable outside the context of the native protein.

A “complementing fragment” is a fragment of a reporter molecule that lacks reporter activity itself, but can functionally reassemble with another complementing fragment to restore reporter activity. Often, the methods and systems of the invention employ enzyme reporter molecules. Accordingly, a complementing fragment pair can functionally reassemble to reconstitute enzymatic activity.

A “member” or “component” of a reporter system refers to a reporter molecule, a fragment or subsequence of a reporter molecule, a subunit of a reporter molecule, or an activator or inhibitor of the reporter molecule.

“Link” or “join” refers to any method of functionally connecting peptides, including, without limitation, recombinant fusion, covalent bonding, disulfide bonding, ionic bonding, hydrogen bonding, and electrostatic bonding. In the systems of the invention, a binding pair member is typically fused, using recombinant DNA techniques, at its N-terminus or C-terminus or both, to a reporter molecule or to an activator or inhibitor of the reporter molecule. The reporter molecule can be a complete polypeptide, or a fragment or subsequence thereof. For example, a binding pair member can be linked to a complementing fragment of a reporter molecule. The binding pair member can either directly adjoin the fragment to which it is linked or can be indirectly linked, e.g., via a linker sequence.

“Fused” refers to linkage by covalent bonding.

A “linker” or “spacer” refers to a molecule or group of molecules that connects two molecules, such as a binding pair member and a complementing fragment of a reporter molecule, e.g., an enzyme, and serves to place the two molecules in a preferred configuration, e.g., so that a fragment of a reporter molecule can interact with a complementing fragment with minimal steric hindrance from a binding pair member and a binding pair member can bind to a binding partner with minimal steric hindrance from the reporter fragment.

“Heterologous”, when used with reference to portions of a protein, indicates that the protein comprises two or more domains that are not found in the same relationship to each other in nature. Such a protein, e.g., a fusion protein or a conjugate protein, contains two or more domains from unrelated proteins arranged to make a new functional protein.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable region light chain (V_{L}) and variable region heavy chain (V_{H}) refer to these light and heavy chain variable regions, respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments can be synthesized de novo, often using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

As used herein, the term “single-chain antibody” refers to a polypeptide comprising a V_{H} domain and a V_{L} domain in polypeptide linkage, generally linked via a spacer peptide (e.g., [Gly-Gly-Gly-Gly-Ser]ₓ), and which may comprise additional amino acid sequences at the amino- and/or carboxy-termini. For example, a single-chain antibody may comprise a tether segment for linking to the encoding polynucleotide. As an example, a scFv is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope). Techniques for the production of single chain antibodies (U.S. Pat. No. 4,946,778) can be adapted to produce antibodies for use in this invention.

The term “expressing components of a selection system” refers to culturing a cell population under conditions in which nucleic acid sequences comprised by expression vectors encoding members of a selection system are expressed.

The term “operably linked” when referring to a nucleic acid, refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

### DETAILED DESCRIPTION OF THE INVENTION

Introduction

The current invention provides a detection system to select improved binding pair members, i.e., binding pair members that have a higher affinity for a cognate binding pair member than that of a reference binding pair member. In the methods and systems disclosed herein, a system comprising four components is used to detect improved binding pair members. The system typically comprises: a host cell, usually bacterial, a library of test binding pair members, each of which is fused to a member of a reporter system, e.g., one of the complementing fragments of a reporter molecule; a cognate binding partner fused to another member of the reporter system, e.g., the other complementing fragment of the reporter molecule; and a “competitor” for binding, which is expressed in the same cells as the fusion proteins to increase the stringency of selection for affinity. The competitor can be any molecule that competes with the binding pair member for binding to the binding partner, but will preferably be a protein, and is typically the antibody or other binding protein one wishes to improve.

The competitor is expressed in the cell at a level such that it binds to most of the cognate binding partner fusion molecules, thereby preventing the binding of test binding pair members that are of equal or lower affinity. The activity of the reporter molecule is reconstituted when testing binding pair members of higher affinity than the competitor are present. For example, in a system employing complementing fragments as reporter members, the test binding pair members of higher affinity bind to the cognate binding partner, thereby providing the complementing fragment to generate reporter activity.

Binding Pairs

Any number of binding pairs are useful in the invention. Often, the binding pairs are polypeptides that specifically interact with one another at discrete binding sites. One member of the binding pair can be incorporated in a fusion protein with one of the complementing fragments of a reporter molecule. The other member of the binding pair can be incorporated into a fusion protein with the other fragment.

In some embodiments, binding pairs are typically antibodies and antigens, but can also be other proteins that have specific binding partners, e.g., interacting subunits of enzymes, receptors and their ligands, proteins which interact in intra-cellular signal transduction and gene regulation, such as the transcription factors c-fos and c-jun, and the like.

Binding partners that involve a member that is not a protein can also be used. For example, small molecule binders may be used by conjugating them to a chemical tag such as biotin. Such conjugates typically can diffuse freely into the bacterial periplasm, allowing them to serve as cognate binding partners to screen for higher affinity test binding partners. For example, test binding partners can be linked to Fragment A and Fragment B can be linked to a protein that binds to the tag, such as avidin or streptavidin for a biotin tag. When the test binding pair member binds to the small molecule cognate binding partner, and the linked tag binds to the tag-binder, the fragments are brought into proximity and the enzyme is activated. In the presence of the competitor, the resulting enzyme activity, and dependent phenotype, will be proportional to the affinity of the test binding pair member, thereby providing the basis for selection of higher-affinity binders of small molecules of interest.

Reporter Systems

A number of reporter systems can be used in the invention. A common type of reporter system is based on complementation of fragments or subunits of the reporter, in which reporter activity is generated from reconstituted complementary fragments or subunits of the reporter. Reporter activity refers to any of a variety of detectable phenotypes, e.g., screenable or selectable phenotypes, such as color, resistance to antibiotics, fluorescence, growth in the presence or absence of particular substrates, and the like. Examples of protein fragments that can be used in a reporter system are provided in WO00/71702. In such a reporter system, fragments pairs reassemble into a marker protein having a detectable signal.

In a complementing fragment pair reporter system, the fragment pair is typically comprised of amino-terminal and carboxyl-terminal fragments of a marker protein. When the fragments are brought into proximity by the interaction of the binding pair members to which the fragments are linked, reporter activity is reconstituted. Enzymes can be particularly useful marker proteins, as there are many enzyme-mediated phenotypic changes that can be used for selection or screening. Enzyme reporters that can be developed into fragments pairs for the reporter systems used in the invention include enzymes that provide for antibiotic resistance, particularly β-lactamase. Other antibiotic resistance enzymes that can be used include aminoglycoside phosphotransferases, such as neomycin phosphotransferase, chloramphenicol acetyl transferase, and the tetracycline resistance protein.

Other proteins that directly elicit a visible phenotypic change such as a color change or fluorescence emission can also be used in generating the complementing fragment reporter system. Example of such proteins include β-galactosidase and green fluorescent protein or other related fluorescent proteins.

The antibiotic resistance protein β-lactamase is often used as the reporter molecule. Particular β-lactamase fragment pairs are exemplified by the α197/ω198 pair. Other β-lactamase can also be derived using techniques described in WO/0071702.

Other reporter systems which confer selectable phenotypes on bacterial cells can also be adapted for cell-based competitive affinity maturation. For example, multimeric reporter proteins with enzymatic or chromogenic activity can be used in a variety of configurations in which the reporter is inactivated by the binding of competitor to cognate binding partner, and re-activated in the presence of test binding pair members having higher affinity for the cognate binding pair member than that of the reference binding pair member. For example, β-galactosidase is a homo-tetrameric enzyme which can produce a selectable color phenotype when expressed in cells in the presence of chromogenic or fluorogenic substrates. Mutant subunits of β-galactosidase have been described which assemble into the active enzyme only when brought together by interaction of heterologous domains fused to the subunits (Rossi et. al., Proc. Natl. Acad. Sci. USA 94:8405-8410, 1997.). When fused to a test binding pair member library and a cognate binding partner, and co-expressed in the presence of a competitor, this β-galactosidase subunit reporter system can be used to select higher-affinity test binding pair members in much the same manner as the β-lactamase fragment complementation system also described herein.

Activation of the reporter occurs when the activity is increased above the level of that observed in a control. Frequently, activation is determined relative to a reference level of activity present in a negative control defined using the reference binding pair member in the detection system. As described in further detail below, the negative control system is designed such that activation of the reporter above the reference level of activity reflects the presence of a binding pair member of higher binding affinity for the cognate binding partner than that of the reference binding pair member.

Competitors

The systems and the methods of the invention include a competitor in the selection system to drive affinity selection. In many embodiments, the competitor is the reference binding pair member, i.e., a known binding pair member for which the practitioner wants to obtain a higher affinity binding analog. As used herein, an analog binds to the same site on a cognate binding partner, e.g., antigen, as the binding pair member, but does not have the identical sequence at its binding site.

As understood by one in the art, cell-based competitive affinity maturation is a reiterative process, wherein the highest-affinity test binding pair member selected in a given round becomes the reference binding pair member for the next round. Generally, the competitor for the next round will be identical to the reference binding pair member. However, the reference binding pair member may be modified for use as competitor by, for example, retaining only the exact binding domain of the reference binding pair member and expressing it without further modification or fused to other domains which may confer desirable properties on the competitor, such as stability.

Competitors can be provided to the cell population in which the affinity maturation occurs in a number of ways. For example, the competitor can be encoded on a separate expression vector or can be included as a discistronic component along with the cognate binding partner fusion protein. Competitors can also be constituitively present in the host cell or otherwise provided, e.g., inducibly expressed.

Generation of Expression Systems Encoding the System Components

Nucleic acids encoding the polypeptides to be expressed in the systems of the invention can be obtained using routine techniques in the field of recombinant genetics. Basic texts disclosing the general methods of use in this invention include Sambrook and Russell, MOLECULAR CLONING, A LABORATORY MANUAL (3rd ed. 2001) and CURRENT PROTOCOLS IN MOLECULAR BIOLOGY (Ausubel et al., eds., John Wiley & Sons, Inc. 1994-1997, 2001 version)).

Often, the nucleic acid sequences encoding the complementing reporter fragments, or binding pair members (or the binding domains of the binding pair members) are cloned from cDNA libraries by hybridization with probes, or isolated using amplification techniques with oligonucleotide primers. Amplification techniques can be used to amplify and isolate sequences (see, e.g., Dieffenfach & Dveksler, PCR Primers: A Laboratory Manual (1995)). Alternatively, overlapping oligonucleotides can be produced synthetically and joined to produce one or more of the domains.

Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Expression Cassettes and Host Cells for Expressing the Fusion Polypeptides, Test Binding Pair Members, and Competitors

There are many expression systems for producing the fusion polypeptides, test binding pair members, and competitors. These systems are well know to those of ordinary skill in the art. (See, e.g., GENE EXPRESSION SYSTEMS, Fernandez and Hoeffler, Eds. Academic Press, 1999) Typically, the polynucleotide that encodes the polypeptide to be expressed is placed under the control of a promoter that is functional in the desired host cell. A variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for expression in a desired host cell.

Fusion polypeptides of the invention can be expressed in a variety of host cells. Often bacterial hosts and expression systems, in particular gram negative bacteria such as E. coli, are employed, but other systems such as yeast, insect, fungal, plant, avian, or mammalian expression systems can also be used. Expression control sequences that are suitable for use in a particular host cell are well known to those of skill in the art. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25) the hybrid trp-lac promoter; the bacteriophage T7 promoter, T3 promoter, SP6 promoter, and the lambda-derived P_{L} promoter and N-gene ribosome binding site (Shimatake et al., Nature (1981) 292: 128).

Phagemid vectors can also be employed, for example, for constructing a library of test binding pair members fused to one of the complementing fragments of the reporter system. Such vectors include the origin of DNA replication from the genome of a single-stranded filamentous bacteriophage, e.g., M13 or f1. A phagemid can be used in the same way as an orthodox plasmid vector, but can also be used to produce filamentous bacteriophage particle that contain single-stranded copies of cloned segments of DNA.

Any available promoter that functions in prokaryotes can be used, although the particular promoter system can be selected for optimal expression as further addressed below. Standard bacterial expression vectors include plasmids such as pBR322-based plasmids, e.g., pBLUESCRIPT™, pSKF, pET23D, λ-phage derived vectors, and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

For expression of fusion polypeptides in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli. These and other suitable bacterial promoters are well known in the art and are described, e.g., in Sambrook et al. and Ausubel et al. Bacterial expression systems for expressing the proteins of the invention are available in, e.g., E. coli, Bacillus sp., and Salmonella (Palva et al., Gene 22:229-235 (1983); Mosbach et al., Nature 302:543-545 (1983). Kits for such expression systems are commercially available.

Either constitutive or regulated promoters can be used in the present invention. Regulated promoters can be advantageous because the host cells can be grown to high densities before expression of the fusion polypeptides is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals.

For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l. Acad. Sci. USA 82: 1074-8). These promoters and their use are discussed in Sambrook et al., supra.

In some applications, eukaryotic expression systems can be used in practicing the methods of the invention. For example, yeast expression systems are well known in the art and can also be used. In yeast, vectors include Yeast Integrating plasmids (e.g., YIp5) and Yeast Replicating plasmids (the YRp series plasmids) and pGPD-2.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells. Inducible promoters for eukaryotic organisms are also well known to those of skill in the art. These include, for example, the metallothionein promoter, the heat shock promoter, as well as many others.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The polypeptides can be expressed intracellularly, or can be secreted from the cell or into the periplasmic space. The expression construct can therefore include sequence, e.g., a leader or signal sequence to allow secretion of the expressed protein.

To facilitate purification of expressed polypeptides, the nucleic acids that encode the fusion polypeptides can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif. ) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

One of skill would recognize that modifications could be made to the reporter domains and binding domains of the expressed polypeptides without diminishing their biological activity. Some modifications may be made to facilitate the cloning, expression, or incorporation of a domain into a fusion protein. Such modifications are well known to those of skill in the art and include, for example, the addition of codons at either terminus of the polynucleotide that encodes the binding domain to provide, for example, a methionine added at the amino terminus to provide an initiation site, or additional amino acids (e.g., poly His) placed on either terminus to create conveniently located restriction sites or termination codons or purification sequences.

Construction of Fission Proteins

The reporter system member, e.g., a complementing fragment of a reporter system, and binding domain of the fusion proteins described herein can be joined directly or indirectly, often via flexible linkers. In a specific embodiment, the coding sequences of each polypeptide in the fusion protein are directly joined at their amino- or carboxy-terminus via a peptide bond in any order.

Alternatively, an amino acid linker sequence may be employed to separate the first and second polypeptide components by a distance sufficient to ensure that each polypeptide folds into its secondary and tertiary structures. Such an amino acid linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Typical peptide linker sequences contain Gly, Ala, Val and Thr residues. Other near neutral amino acids or polar residues that have heteroatoms such as Ser and Met can also be used. Amino acid sequences which may be usefully employed as linkers include those disclosed in Maratea et al. (1985) Gene 40:39-46; Murphy et al. (1986) Proc. Natl. Acad. Sci. USA 83:8258-8262; U.S. Pat. Nos. 4,935,233 and 4,751,180. The linker sequence may generally be from 1 to about 50 amino acids in length, e.g., 3, 4, 5, 6, or 10, 15, 20, 25, 30, 35, 40, 45, or more amino acids in length. Often, the linker is 15 amino acids in length. Linker sequences may not be required when the first and second polypeptides have non-essential N-terminal amino acid regions that can be used to separate the functional domains and prevent steric interference.

Although most frequently a binding partner is a protein, it does not have to be a protein. Thus other fusion methodologies can also be employed. Other methods of joining the domains include chemical conjugation, methods such as ionic binding by expressing negative and positive tails, and indirect conjugation through means such as streptavidin-biotin interactions. (See, e.g., Bioconjugate Techniques, supra). For example, small molecules can be chemically conjugated to biotin via N-linked, O-linked, or S-linked spacers, and such conjugates can be readily diffusible into the periplasm form the medium. The binding protein library fused to the α197 fragment can then be co-expressed with avidin fused to the ω198 fragment, such that binding of the biotin moiety on the antigen to the avidin-ω198 fusion will complement any binder-antigen interaction to drive reconstitution of active β-lactamase, thereby allowing growth of the cells expressing the antigen binder in the presence of the antibiotic. The domains can also be joined together through an intermediate sequence.

Library of Test Binding Pair Members

A library of test binding pair members that is to be used for selection of an improved binding pair member can be generated using a number of vectors and methods known in the art. The library can be expressed using any number of vectors, such as those described above. Often, the library vector is a phagemid.

The test binding pair member, which is typically fused to one of the members of the reporter system, e.g. a complementing fragment, is often an antibody. An expression library therefore can include DNA sequences that encode the epitope-binding portions of heavy- and light-chain variable regions of immunoglobulin (Ig); see, e.g., Marks, J. Biol. Chem. 267: 16007-10, 1992; Griffiths, EMBO J. 12: 725-734, 1993. Alternatively, the displayed protein can be a single-chain (scFv) Ig fragment (see, e.g., Pistillo Exp. Clin. Immunogenet. 14:123-130, 1997.

In generating a library of test binding pair members, the binding domain of the best binding pair member typically does not differ from the binding domain of the reference binding pair member by more than a few mutations. Otherwise, a test binding pair member may be selected by non-competitive binding to the cognate binding partner rather than by having a higher affinity. Accordingly, the binding domain of the test binding pair members, typically 80-250 amino acids in length, is often greater than 90%, e.g., 95%, 96%, 97%, 98%, or 99% identical to the binding domain of the reference binding pair member over a comparison window of 25 amino acids, optionally about 50-100 amino acids or the entire length of the binding domain.

The sequence can be compared and aligned for maximum correspondence over a comparison window, or designated region as measured using one of the following sequence comparison algorithms or by manual alignment and visual inspection. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The comparison window includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands. For purposes of this patent, percent amino acid identity is determined by the default parameters of BLAST.

The library can be a randomly generated library of test binding pair members. Often the library is a mutagenized library. The library of test binding pair members can be generated using a variety of mutagenesis methods including, for example, error-prone PCR (Cadwell and Joyce, in PCR Primer, A Laboratory Manual, Dieffenbach and Dveksler, Eds. Cold Spring Harbor Press, Cold Spring Harbor N.Y., pp. 583-590, 1995), Parsimonious Mutagenesis (PM) (Balint and Larrick, Gene 137:109-118, 1993), DNA shuffling (Crameri et al., Nature Biotechnol. 14:315-19, 1996), random-priming recombination (RPR) (Shao et al., Nucleic Acids Res. 26:681-683, 1998), or the staggered extension process (StEO) (Zhao et al., Nature Biotechnol. 16:258-261, 1998). For these methods, except PM, it is typically desirable to have a mutation rate of 1-3 mutations per clone to avoid unwanted mutations. For PM, higher mutation rates can be used because only the protein combining sites of the binder are mutagenized. PM may therefore be advantageous for accessing larger affinity increments. PM has additional advantages in avoiding folding mutants, avoiding immunogenicity, and ease of sequencing.

Culture Conditions for Affinity Selection

The affinity maturation procedure disclosed herein takes place in a bacterial cell, typically, a gram negative bacterium. Selection is desirably performed under optimized conditions. Optimization can be performed by considering a number of factors, including the optimization of the negative control.

A negative control is established, which negative control is typically the maximum affinity one wishes to exclude from selection, i.e., that of the binder to be improved. Thus, for the negative control, the binding pair member to be improved is expressed not solely as the competitor but also as the reference binding pair member, in place of the test binding pair members. Several conditions are generally taken into consideration for optimal selection: (1) the cognate binding partner-reporter fragment fusion is limiting, preferably no more than about one-tenth the concentration of the competitor, in order to force competition between the competitor and the test binding pair members, (2) the competitor is in excess over a concentration equal to its K_{d}, preferably 10-fold, so that the cognate binding partner/reporter fragment fusion is at least 90% competitor-bound in the negative control, and (3) the test binding pair member-reporter fragment fusion concentration should be comparable to that of the cognate binding partner, i.e., no more than about 10% that of the competitor, so that the negative control has not more than about 10% of the maximum reporter activity. This provides a dynamic ranges of a factor of about 10.

Control of Levels of Expression of System Components

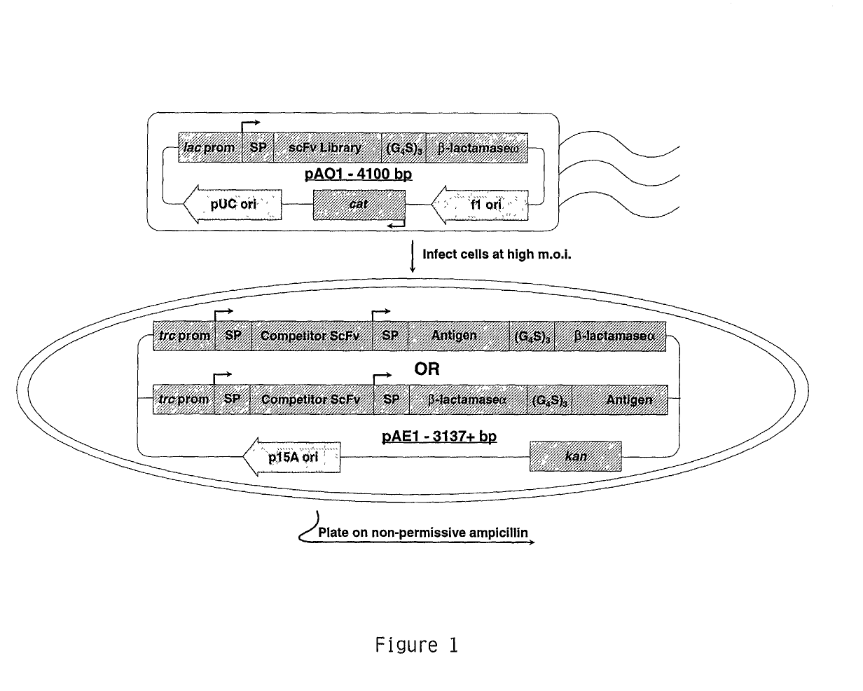

In practicing the methods of the invention, the levels of expression are typically controlled to achieve desirable relative levels of expression of the components of the system. Suitable conditions can be achieved, for example, by expressing three components of a selection system as illustrated in FIG. 1. The competitor and the cognate binding partner/reporter fragment fusion, e.g., an antigen-α fragment (of β-lactamase) fusion, can be expressed from a dicistronic transcript from a strong promoter such as the trp-lac fusion promoter. Translation of the upstream cistron is typically more efficient than that of the downstream cistron. In such conditions, a competitor encoded by the upstream cistron would be present in excess and a cognate binding partner/reporter fragment fusion would be limiting. The binding pair member, e.g., antibody, fused to the complementing reporter fragment, e.g., an ω fragment of β-lactamase, can then be expressed from a weaker promoter such as the lac operon promoter in a separate, compatible vector. This will produce the binding pair member/complementing reporter fragment fusion in amount comparable to that of the cognate binding partner/reporter fragment fusion.

If such expression conditions are not optimal for a given antigen-antibody pair, additional manipulations are available to further control the expression levels of one or more of the components. For example, if competitor expression is weak, stronger promoters, e.g., the bacteriophage T7 promoter, are available. Alternatively, competitor expression, and also expression of a binding partner can be improved without compromising functionality using other means, e.g., Fold Selection technology as disclosed in U.S. patent application No. 09/510,097.

If expression of any component is too strong, or if proportions among the components are not suitable, inducible promoters can be used, such as the arabinose operon promoter, which allows the expression level of any of the components to be manipulated to an appropriate level. For example, antigen fusion or scFv fusion expression could be reduced to 0.1× the competitor, or competitor expression could be increased to 10× a concentration equivalent to its K_{d}.

In order to evaluate overall levels of expression of the components of a system, the amount of polypeptide produced by the expression vectors can be determined using assays such as immunoblots. In practice, the levels of expression of the components of the system are established empirically in a negative control based on the considerations identified above.

Antibody Selection and Optimization for Affinity Maturationy

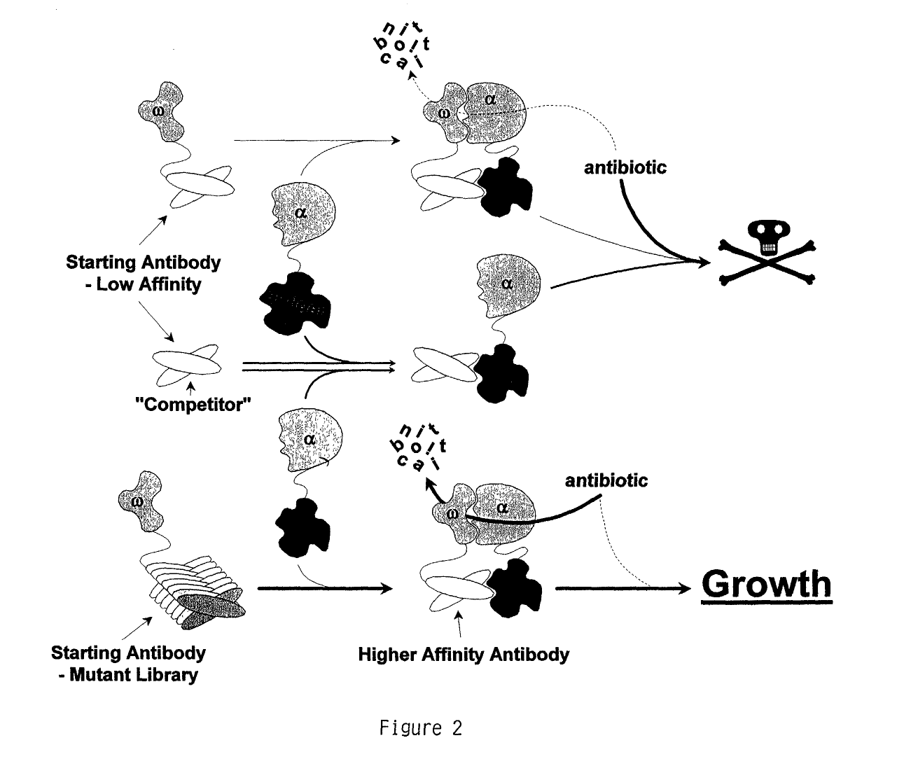

A general example of use of the invention for antibody selection and affinity maturation is provided in FIG. 2. This general example uses a β-lactamase complementation fragment reporter system When the two fusion protein expression vectors are expressed together in the same cells, the antigen-antibody interaction brings the β-lactamase fragments into close proximity, and this facilitates refolding of the fragments into the active enzyme. The resultant β-lactamase activity allows the cells to grow in the presence of β-lactamase antibiotics at concentrations that quantitatively kill cells expressing non-binding antibody-antigen pairs. A third component, a competitor, can be introduced into the cell to increase the stringency of selection for affinity. The competitor can be any molecule that competes with the antibody for binding to the antigen, but is typically the antibody to be improved or an analog thereof.

In this example, the β-lactamase fragment pair designated α197 (amino acids H26-E197) and ω198 (amino acids L198-W288), enhanced with the break-point resealing peptides NGRE and QGN at the α and ω break-points, respectivley, are used. This fragment pair and others that can be used in the methods of the invention are described, e.g., in WO00/71702.

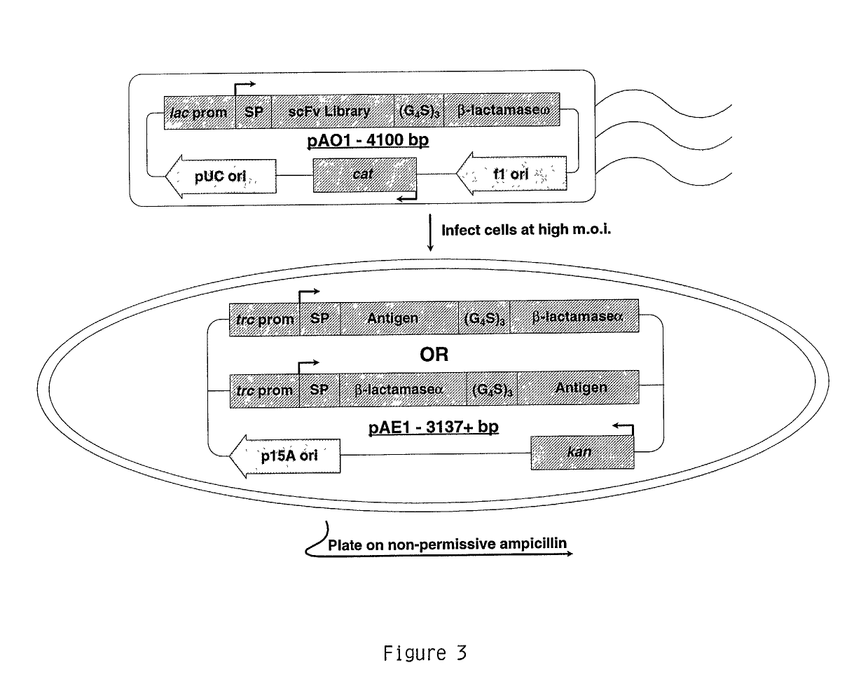

Antibodies in the form of single-chain Fv fragments (scFv) (e.g., Marks et al., Eur. J. Immunol. 21:985-991, 1991) may be expressed as fusions to the amino terminus of the ω198 fragment, e.g., via intervening (Gly₄Ser)₃ linkers. Antigens may also be expressed as fusions to the carboxy (break-point) terminus of the α197 fragment via intervening (Gly₄Ser)₃ linkers. The scFv-ω198 fusion may be expressed from a plasmid vector such as pAO1, a pUC phagemid vector expressing chloramphenicol resistance and containing a cassette for expression of inserted coding sequences as ω198 fusions from the lac operon promoter. The α197-antigen fusions may be expressed from such a pUC-compatible plasmid vector as pAE1, a p15A replicon expressing kanamycin resistance and containing a cassette for expression of inserted coding sequences as α197 fusions from the trp-lac fusion promoter (Sambrook, supra). These expression constructs for antibody selection are illustrated in FIG. 3.

Typically, sequences encoding a library of test binding pair members, e.g. a diverse populations of scFv, are subcloned into a vector, such as the pAO1 vector, transfected into an appropriate E. coli host strain, such as TG-1, and rescued as filamentous bacteriophage by super-infection with the helper phage M13K07 (see, FIG. 3). The resulting phage population is then used to quantitatively infect cells harboring the pAE1 vector expressing the antigen of interest as the α197 fusion.

The number of cells infected is usually at least ten-fold the size of the scFv library to ensure screening of the entire library. If the number of phage used is at least ten-fold the number of cells, and the phage concentration is at least 1×10¹² transforming units per ml, then most cells will be infected by more than one phagemid, which also provides comprehensive exposure of the antibody library to the antigen.

Under these expression conditions, the concentrations of the β-lactamase fragment fusion proteins in the E. coli periplasm are expected to be in the range 0.1-10 μM, which is equivalent to 10-1000 molecules per cell. Thus, antibody affinities in the micromolar K_{d} range should produce roughly 10-90% activation of β-lactamase, or in the range of 10-90 molecules of active β-lactamase per cell. Immunoblot analysis of soluble protein has shown that as few as 10 molecules per cell of reconstituted β-lactamase is sufficient for quantitative plating on 50 μg/ml ampicillin, and that the plating efficiencies for non-interacting fusion proteins is <10⁻⁶ on the same concentration of ampicillin. This means that micromolar affinities should be readily selectable, but that the system will not be able to discriminate readily among higher affinities, even if higher concentrations of ampicillin are used, because maximum β-lactamase activation will occur at K_{d}'s of 1-100×10⁻⁸M. As the TG-1 strain of E. coli expresses the lac promoter repressor (lacl^{q}) gene, expression of both antibody and antigen fusion are expected to be repressed by at least a factor of 10 in the absence of the lac promtoer inducer IPTG relative to the fully-induced state, so that the upper limit of selectable K_{d}'s in the absence of IPTG is in the range 1-10×10⁻⁸ M.

Most human germline heavy chain variable region (V_{H}) genes do not express well in E. coli, especially when the cells are grown continuously under selection pressure. However, a few germline V_{H} genes, such as DP47, are expressed well naturally and can be used as platforms for libraries of diverse binding specificities when the sequence of the third complementarity-determining regions (CDR3) is randomized, and the V_{H} library is coupled with a light chain variable region (VL) library based on a compatible V_{L} germline gene such as DPL3 or on a V_{L} repertoire library. In addition, other germline V_{H}s can be stabilized with usually one or two mutations using technology such as Fold Selector technology (see, e.g., U.S. patent application Ser. No. 09/510,097.

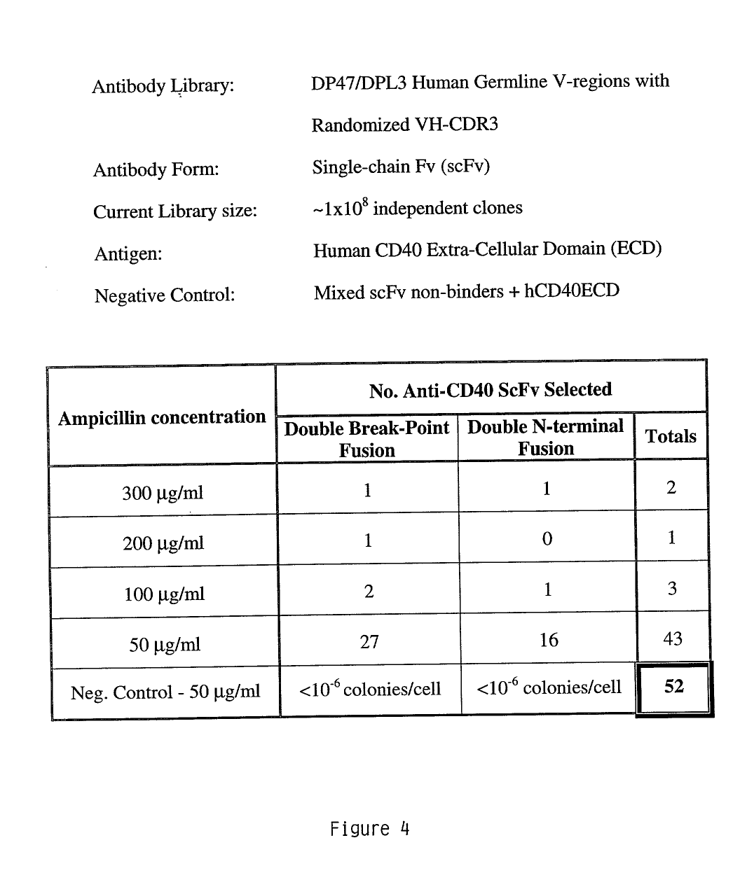

When a library of about 10⁸ human scFv based on the DP47 germline VH gene and the DPL3 germline V_{L} gene with V_{H} CDR3s containing 12-16 amino acids of random sequence was fused to the ω198 fragment and quantitatively co-expressed with the extra-cellular domain of the human B-cell antigen CD40 fused to either end of the α197 fragment in TG-1 cells in the absence of IPTG, a total of 52 CD40-specific scFv were obtained on various concentrations of ampicillin. It can be inferred that the scFv which confers quantitative resistance to a maximum of 50 μg/ml ampicillin should have K_{d}s <10×10⁻⁸ M, and the scFv that confer resistance to >200 μg/ml ampicillin should have K_{d}s <10×10⁻⁹ M (FIG. 4).

An example of affinity maturation using β-lactamase is provided in FIG. 2. Before affinity selection, conditions are established for the desired stringency of selection. This involves determining the non-permissive ampicillin concentration for cells expressing the negative control. The negative control will be the maximum affinity one wishes to exclude from the selection, i.e., the antibody to be improved. Thus, for the naegative control the binder to be improved is expressed not just as the competitor but as a fusion as well (see, example in FIG. 2). For example, in accordance with the considerations noted above, if a 1 μM K_{d} competitor is at 10 μM in the bacterial periplasm (about 1000 molecules per cell) with μM antigen-α fusion and μM binder-ω fusion, then about 90% of the antigen will be bound and about 10% of that will be bound by the binder-co fusion and be activated. A test binding pair member with a 10-fold higher affintiy would then increase the β-lactamase activity about 5-fold to about 50% of maximum, and a 100-fold increase in affinity would raise activity to about 90% of maximum. Such increases in activity can be identified by increased plating efficiency in the presence of ampicillin. This means that for efficient recovery (in this case on a solid medium) of test binding pair members with 10-fold higher affinities, the selective ampicillin concentration should not be higher than that needed to give a plating efficiency of 10⁻³-10⁻⁴ for the negative control. In general, to ensure efficient capture of higher-affinity antibodies, the number of transformed cells plated is equivalent to about ten times the inverse of the product of the minimum expected plating efficiency and the minimum expected frequency.

If the frequencies of 10-fold affinity mutants in the library are less than the plating efficiency of the negative control, false positive may outnumber true positives, in which case it may be necessary to replate the selected colonies to eliminate the false positives. In general, for efficient capture of higher-affinity antibodies, the number of transformed cells plated should be equivalent to ten times the inverse of the product of the minimum expected plating efficiency and the minimum expected frequency. For example, if the expected frequency is 10⁻⁴ and the expected plating efficiency is 10%, then at least 10⁶ cells are plated.

As appreciated by one of skill in the art, selection of higher-affinity mutants can also be achieved by growth in suspension culture. As noted above, under optimum expression conditions, multiple replatings on solid medium may be required to eliminate false positives. However, the required enrichments could be achieved in a single 1-2 day period of competitive growth in suspension culture, at the end of which more stringent plating conditions, i.e., higher antibiotic concentrations, can be used to eliminate the false positives because quantitative plating is not required for efficient recovery of the enriched higher-affinity mutants.

For enrichment of higher affinity binding pair members by competitive growth in suspension, the ampicillin concentration is first adjusted to allow only slow growth of the negative control. This ensures that the stringency is not too high for small affinity increments, but that small affinity increments should still be able to enrich rapidly. Once the optimum stringency has been determined, the antigen-binding protein is mutagenized for expression as the ω-fragment fusion. The binder coding sequence can be mutagenized by any of a variety of methods, supra.

The affinity selection process is typically initiated with parallel suspension cultures of the negative control and the mutagenic library in an appropriate E. coli strain such as TG-1 in a standard medium such as L Broth. The cell concentration and ampicillin concentration are desirably set to allow the negative control to double no more than a few times in an overnight period to reach an OD₆₀₀ of no more than 0.01-0.1. Under such conditions, a modest affinity increment can produce a larger increase in growth rate so that even at a low frequency, the culture density of the library could be detectably higher after overnight growth. For example, for a library of 10⁷ variant clones, 100 ml starting culture of about 10⁶ cells/ml in an ampicillin concentration that would allow the negative control to double every 4 hours could be used to initiate selection. After 16 hours, the control culture would have a concentration of about 1.6×10⁷ cells per ml (about 0.016 OD₆₀₀), whereas the mutant culture would have a concentration of about 2.3×10⁷ cell per ml (about 0.023 OD₆₀₀) if all mutants producing an average 4-fold increase in the growth rate had a combined frequency of at least 10⁻⁴ in the library.

The growth rate typically scales non-linearly with affinity, such that a 4-fold increase in the growth rate corresponds to a <4-fold increase in affinity. Thus, after 16 hours of growth, more than 25% of the cells in the mutant culture would have affinities averaging-fold higher than that of the competitor. At this point, equal number of cells are plated from both cultures onto solid medium under conditions where the control culture background would be zero, e.g., 10⁴ cells are plated onto an ampicillin concentration on which the negative control has a plating efficiency of 10⁻⁴. The same number of cells from the mutant culture produces many colonies with a diversity of mutations. Each of these clones is then tested on higher concentration of antibiotic to determine which clones have the highest affinities. Under optimal conditions, the dynamic range should allow discrimination of up to a 100-fold increase in affinity, if such variants are present, producing up to a 10-fold increase in β-lactamase activity.

After the first aliquots are removed, the cells are centrifuged and resuspended in fresh medium containing fresh antibiotic. Aliquots can then be taken as frequently as desired and plated as above. The mutant culture in this example approaches stationary phase (about 10⁹ cells per ml) after 7-8 hours of incubation, at which point the control culture will have only gone through about 2 additional doublings to about 3×10⁷ cells per ml. thus, only about 3% of the cells in the stationary phase culture of the mutant library are expressing unimproved antibodies.

The diversity of the highest affinity mutants in the stationary phase culture can be determined by first plating for zero background as described above, and then by re-plating recovered clones onto successively higher antibiotic concentrations. The genetic diversity of the most antibiotic resistant clones is then determined by sequencing. One or more of these can be used as the competitor in the next round of mutagenesis and selection. Subsequent rounds of diversification can be accomplished by mutagenesis, or by recombining mutations selected at any point in the previous rounds of selection. This process is typically repeated until no new mutations are isolated, i.e., when the highest affinity variant is used as competitor and continues to dominate the selection among other variants.

Selection of higher-affinity antibodies can potentially be compromised by the selection of stable phenotypic revertants, i.e., cells that have acquired mutations that confer antibiotic resistance without increasing the affinity of the antibody. Normally, reversion frequencies involving gain-of-function mutations are low enough relative to desired mutant frequencies to pose no significant problem for simple selection systems such as the β-lactamase fragment complementation system. However, the competitive affinity selection system can be subverted more readily by loss-of-function mutations in the competitor gene. Since the competitor is on a separate plasmid from the antibody library, which is on a phagemid, revertants can be easily eliminated by rescuing the selected mutant antibody phagemids with helper phage, and re-infecting fresh competitor/antigen-expressing cells. Antibodies that were originally selected by virtue of loss of the competitor will not be reselected in the presence of fresh competitor, while true higher-affinity mutants should be efficiently re-selected. Possible non-affinity revertants can also be eliminated by sub-cloning the coding sequences of selected antibodies before selecting a second time.

Not to be bound by theory, the selection is expected to be driven by affinity until the lifetimes of antigen-antibody complexes become long compared to the cell doubling times, at which point selection becomes driven primarily by the association rates, on-rates, of the antibody-antigen complexes. On-rate selection in a heterologous protein-rich environment such as the bacterial periplasm biases the selection toward rigidification by the CDRs, by which the entropy cost of binding is reduced, and toward increased CDR-epitope surface complementarity, by which the proportion of productive encounters is increased. The latter effect is also expected to increase the binding energy by increasing proximity-induced van der Waals interactions, which with increasing surface complementarity can become the dominant component of binding energy. This effect increases the off-rate as well, thus producing a balanced increase in affinity. Thus, the upper limit of affinities that can be discriminated, and therefore selected, is determined by the diffusion limit for globular proteins in the about 50 kDa size range in the bacterial periplasm. Assuming that such a limit is not less than about 10⁷M⁻¹sec⁻¹, and that associated off-rates are as low as 10⁻⁵sec⁻¹, antibodies with affinities in the nanomolar to picomolar K_{d} range can be obtained by this method.

Uses of the Methods and Systems of the Invention

Affinity maturation can be used in a number of applications to generate higher affinity binding pair members. For example, the methods and systems of the invention can be used to generate superior antibodies or human antibodies corresponding to a mouse counterpart. The techniques can also be used to identify peptides that have a higher binding affinity, e.g., peptides that are improved agonists or antagonists for a receptor, or for any other application for which a binding peptide with enhanced affinity is desirable. As noted above, small molecules with enhanced binding affinity for a target can also be identified, e.g., using a biotin tag system.

Therapeutic Antibody Development

In one application, the systems and methods of the invention can be used to develop therapeutic antibodies. Affinity maturation can mold low-affinity antibody combining sites into rigid shapes with high complementarity to epitope surfaces. Therefore, the starting libraries do not have to be particularly large or diverse. For example, a library can be built on a single-chain Fv platform comprising a single pair of well-expressing human germline VH and VL regions with random sequences inserted into the CDR3 of VH and VL. Such a library can be made efficiently by ligating synthetic oligonucleotides containing random sequences to appropriate restriction endonuclease sites engineered into the antibody coding sequence. A library of about 10⁸ such sequences typically has enough diversity to produce antibodies of micromolar affinity in the methods of the invention.

An additional advantage of a single-platform library is that the antibody expression levels are typically uniform. Structural diversity among the antibodies is limited to the surface of the protein, and therefore has little impact on either the folding kinetics of the stability of the antibodies. Furthermore, e.g., in the PM method for mutagenesis, mutations can be limited to the CDRs and are therefore less likely to affect expression levels.

For therapeutic applications, important performance parameters for antibodies include specificity, stability, lack of immunogenicity, and the on-rate. For most antibody targets in vivo the off-rate does not need to be lower than 10⁻³-10⁻⁴sec⁻¹, which corresponds to half-lives of 11 min to 2 hours. Most surface-bound antibodies either undergo endocytosis or engage in effector functions such as phagocytosis or complement fixation within this time frame. However, there is no identified upper limit beyond which further increases in the on-rate are not therapeutically advantageous. Thus, the invention described herein can provide superior antibodies for therapeutic applications. If it is advantageous to use full-length antibodies of specific isotypes for therapeutic applications, linkers present in affinity matured antibodies can be removed and the required constant regions added to the scFvs.

### EXAMPLES

### Example 1

Competitive Determination of Complementation Groups Among Thioredoxin-Scaffolded Peptides that Bind Human CD40

In addition to antibodies, other types of protein scaffolds for binding domains, particularly those which can be expressed intracellularly, can also be used. For example, random peptides of up to 20 amino acids can be inserted into the active site of bacterial thioredoxin (trxpepes), and antigen-binding molecules can be selected from such libraries (see, e.g., Colas et al., Nature 380:548-550, 1996). This example uses the β-lactamase α197/ω198 fragment complementation system to select a panel of 12-amino acid trxpeps for binding to a cell-surface antigen, the extracellular domain of the human B-cell activation antigen CD40, which can be expressed in the E. coli periplasm (see, e.g. Noelle et al., Immunol. Today 13:431-433, 1992 and Bajorath & Aruffo, Proteins: Struct, Funct., Genet. 27:59-70, 1997).

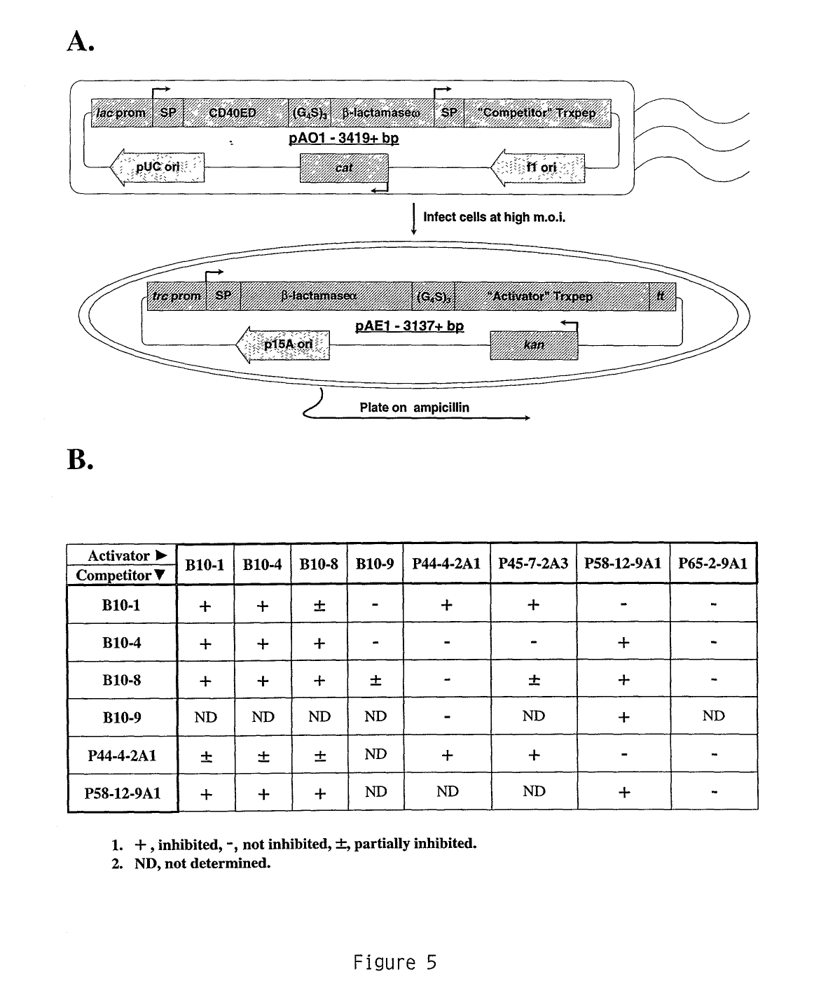

The coding sequence for the mature form of the extracellular domain (CD40ED) was amplified by PCR using primers homologous to the N-terminus of the mature protein and to the C-terminus of the about 190-residue extracellular domain (Genbank accession no. X60592). The PCR product was subcloned into the pAO1 phagemid vector (FIG. 5A) for expression from the lac promoter as a C-terminal fusion to the β-lactamase ω198 fragment with an intervening (Gly₄Ser)₃ linker. Expression of the correct product was confirmed by polyacrylamide gel electrophoresis (PAGE), and the CD40 fusion vector was then rescued as phage and transfected into TG-1 cells bearing the Trxpep library construct. A commercial Trxpep library was obtained and amplified by PCR using primers specific for the N- and C-termini of E. coli thioredoxin (Genbank accession no. M54881). This product was subcloned into a p15A replicon (Rose, Nucleic Acids Res. 16:355, 1988) for expression as fusions to the C-terminus of the α197 fragment from the trp-lac fusion promoter (pAE1, FIG. 5A). About 10⁷ co-transformants were collected by double selection on kanamycin and chloramphenicol, and then plated onto 25 μg/ml ampicillin.

Ampicillin-resistant clones encoding thirteen unique trxpeps were recovered. In all cases, ampicillin resistance was strictly dependent on the presence of CD40ED and the peptide portion of the trxpep. No activity was seen if CD40ED was replaced with an irrelevant protein or if the trxpep was replaced by wild-type thioredoxin. Inter-trxpep competition was tested by expressing each of five selected CD40-binding trxpeps as a “competitor” from a second cistron in the pAO1 phagemid vector, downstream from the CD40-ω198 fusion (FIG. 5A). Each of these constructs was then co-expressed with each of the same five plus three additional selected α197-trxpep fusion constructs in strain TG1 and scored for growth on 25 μg/ml ampicillin. The results are shown in FIG. 5B.

In each case tested, activation of β-lactamase by the binding interaction between CD40 and trxpep was strongly inhibited in the presence of the same trxpep as competitor. Thus, an alteration in the α197-fused trxpep that increased its affinity for CD40 should be able to counteract the effect of the competitor and selectably restore at least some of the lost β-lactamase activity. The eight trxpeps sorted into five complementation groups, corresponding approximately to five different epitopes. The trxpeps p58-12-9A1, BW10-4, and BW10-8 comprise one group, competing strongly with each other and having similar competition profiles. Thus, it was concluded that these three trxpeps were competing for the same epitope on CD40.

When cells expressing each of the three trxpeps as the α197 fusion were grown in suspension culture in the presence of 25 ug/ml ampicillin, all three doubled at about the same rate during log-phase growth. However, when each trxpep was co-expressed as a free competitor with each of the α197-trxpep fusions, the suspension growth rates in 25 μg/ml ampicillin were inhibited to different extents, although the pattern was always the same. Cells expressing α197-BW10-8 fusion always grew fastest, regardless of the competitor, followed by the α197-p58-12-9A1 fusion, followed by the α197-BW10-4 fusions. As the expression levels of all three α197-trxpep fusions were comparable, as judged by immunoblots of soluble extracts, the growth rates under competitive conditions correlate with affinity for CD40. Thus, BW10-8 has the highest affinity for CD40, followed by p58-12-9A1, followed by BW10-4.

Cells expressing each of the three α197-trxpep fusions and BW10-4 as the competitor were mixed in equal numbers and allowed to grow overnight in suspension culture in the presence of 25 μg/ml ampicillin. Of 20 clones selected at random from the resulting cultures, all 20 expressed the p58-12-9A1 trxpep as the α197 fusion. Thus, the highest affinity variants can be selected by competitive growth in suspension culture.

### Example 2

Affinity Competition Between an Anti-CD40 Antibody and a Higher-Affinity Mutant of the Same Antibody

This example shows the ability of the competitive affinity selection system to discriminate between closely related antibodies with affinities (K_{d}) in the nanomolar range.

The 5D12 antibody is a murine monoclonal antibody specific for human CD40. The Fab fragment of this antibody has a K_{d} of 7.6 nM. A variant of this antibody (5D 12-6D) with two mutations in CDR3 of V_{H} has a K_{d} of 0.64 nM. The plating efficiencies of these two antibodies under competitive an non-competitive conditions are listed in Table I.

When the scFv of 5D12 was expressed as the ω198 fusion in the same cells as the α97 fusion of CD40 extracellular domain (CD40ED), the cells were quantitatively resistant to only 10 μg/ml ampicillin. However, when the 5D12-6D mutant was expressed in the same system, the cells plated quantitatively out to 25-50 μg/ml. The fact that the system could discriminate between these two antibodies indicates that the antibodies were unstable and had apparent affinities that were much lower than those of the Fabs. Normally the system should not be able to discriminate nanomolar affinities because to do so would require sub-nanomolar concentrations of the fusions and such concentration in the bacterial periplasm would be equivalent to less than one molecule per cell.

To restore the affinity of the 5D12 Fab to the 5D 12scFv, it was necessary to stabilize it expression it the bacterial periplasm. The principal sources of instability are typically aggregation-prone folding intermediates and/or loose association of V_{H} and V_{L} due to the length of the flexible linker. Both deficiencies can be repaired by selecting mutations that destabilize the folding intermediates and thereby accelerate folding. The same mutations also usually increase the affinity between the two chains of the scFv. When the stability of the SD12scFv was mutationally restored, the stabilized scFv (s5D12) conferred quantitative plating out to 100-200 μg/ml ampicillin (see, Table I). When the same stabilizing mutations were introduced into 5D12-6D, however, the plating efficiency did not increase significantly beyond the level of s5D12, probably due to the fact that the two steady state concentrations of both antibodies in the periplasm were far higher than their affinities (K_{d}), so that the difference in the latter couldn't be detected, i.e., when the concentration is greater than the K_{d}, affinity is not longer limiting for activity.

The two stabilized antibody-co fusions were then co-expressed with the α-antigen fusion in the presence of s5D12 as the free competitor. As shown in Table I, the presence of the competitor reduced the plating efficiency of s5D12 from about 50% on 200 μg/ml ampicillin to about 50% on 25 ug/ml. On 200 μg g/ml ampicillin, the competitor reduced the s5D12 plating efficiency to <10⁻⁵ (not shown). The same competitor reduced the plating efficiency of the 6D mutant from about 70% on 200 ug/ml to about 40% on 100 μg/ml. An important observation is that on 100 μg /ml ampicillin in the presence of the competitor, the s5D 12-6D mutant plated with at least 1000-fold higher efficiency than its parent antibody, s5D12, whereas in the absence of the competitor the two could barely be distinguished at the same concentration. Thus, use of the competitor allowed a 12-fold increment in affinity to confer at least a 1000-fold increment in selectability. Accordingly, after each round of plating on 100 μg/ml ampicillin, the frequency of the mutant should be enriched at least 1000-fold relative to the parent. This experiment therefore demonstrates the ability of the competitive affinity selection technique to discriminate between affinities at the nanomolar K_{d} level.

TABLE I
Correlation Between Growth and Affinity for two Anti-CD40 Antibodies with Nanomolar K_{d}s

| ScFv ^{a.} | K_{d} | Antigen | Competitor | Amp10 ^{b.} | Amp25 | Amp50 | Amp100 | Amp200 |
|---|---|---|---|---|---|---|---|---|
| 5D12-ω | 7.6 × 10⁻⁹ M | α-CD40ED | — | 75 | 13 | <0.01 | <0.01 | <0.01 |
| 5D12-6D-ω | 6.4 × 10⁻¹⁰ M | α-CD40ED | — | 75 | 100 | 50 | 6 | <0.01 |
| 5D12-ω | 7.6 × 10⁻⁹ M | α-CD40ED | 5D12 | 5 | <0.01 | <0.01 | <0.01 | <0.01 |
| 5D12-6D-ω | 6.4 × 10⁻¹⁰ M | α-CD40ED | 5D12 | 75 | 5 | <0.01 | <0.01 | <0.01 |
| s5D12-ω | 7.6 × 10⁻⁹ M | α-CD40ED | — | 100 | 100 | 100 | 13 | 0.5 |
| s5D12-6D-ω | 6.4 × 10⁻¹⁰ M | α-CD40ED | — | 100 | 80 | 100 | 20 | 2 |
| s5D12-ω | 7.6 × 10⁻⁹ M | α-CD40ED | s5D12 | 75 | 5 | <0.01 | <0.01 | <0.01 |
| s5D12-6D-ω | 6.4 × 10⁻¹⁰ M | α-CD40ED | s5D12 | 100 | 100 | 40 | 2 | <0.01 |

^{a.} 5D12, anti-CD40 antibody; s5D12, stabilized anti-CD40 antibody; s5D12-6D, stabilized higher-affinity mutant (2 mutations) of the 5D12 anti-CD40 antibody.
^{b.} Amp10, Amp25, Amp50, etc., refers to the plating efficiency on 10, 25, 50, etc., μg/ml. ampicillin. The plating efficiency is equal to the percentage of doubly-transformed cells which form colonies.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A competitive method of identifying a test binding pair member with a higher affinity for a cognate binding partner than a reference binding pair member, the method comprising:
(a) expressing components of a selection system in a population of bacterial cells, the selection system comprising:
one or more molecules of a reporter system;
a library of test binding pair members;
a cognate binding partner; and
a competitor that has the properties of: i) competing with the reference binding pair member for binding to the cognate binding partner and ii) having an affinity for the cognate binding partner at least equal to that of the reference binding pair member;
wherein one or more molecules of the reporter system is uniquely linked to one or more of the following components: the library of test binding pair members, the cognate binding partner, or the competitor;
(b) culturing the population of cells under conditions in which the reporter system is activated when a test binding pair member having a higher affinity than the competitor binds to the cognate binding partner, thereby conferring a selectable phenotype on the cells; and
(c) selecting a cell exhibiting the selectable phenotype.

2. A competitive method of identifying a test binding pair member with a higher affinity for a cognate binding partner than a reference binding pair member, the method comprising:
(a) introducing an expression vector comprising nucleic acid sequences encoding a library of test binding pair members linked to a fragment A of a marker into a population of bacterial cells;
(b) introducing into the population of cells an expression vector comprising nucleic acid sequences encoding the cognate binding partner linked to a fragment B of a marker;
wherein the marker is active when the fragment A and the fragment B are in proximity;
(c) culturing the population of cells under conditions in which the library of test binding pair members linked to fragment A and the cognate binding partner linked to fragment B are expressed in the presence of a competitor that has the properties of:
i) competing with the reference binding pair member for binding to the cognate binding partner; and
ii) having an affinity for the cognate binding partner at least equal to that of the reference binding pair member; wherein a test binding pair member having a higher affinity than the competitor binds to the cognate binding partner linked to fragment A; and
(d) selecting a cell in which the marker is active.

3. The method of claim 2, wherein the binding domain of the test binding pair member is 90% identical to the binding domain of the reference binding pair member.

4. The method of claim 2, wherein the selecting step comprises selecting a cell in which the marker is more active than a reference standard of activity.

5. The method of claim 2, wherein the competitor is the reference binding pair member.

6. The method of claim 2, wherein the reference binding pair member is an antibody.

7. The method of claim 6, wherein the antibody is a single chain antibody.

8. The method of claim 7, further wherein the test binding pair members linked to fragment A are single chain antibodies.

9. The method of claim 2, wherein the cognate binding partner linked to fragment B is expressed at a concentration that is limiting, the competitor is expressed in an amount that is in excess over a concentration equivalent to its K_{d} for binding to the cognate binding partner, and the concentration of the test binding pair member linked to fragment A expressed in the cell population is substantially the same as that of the cognate binding partner linked to fragment B.

10. The method of claim 9, wherein the concentration of the cognate binding partner linked to fragment B is one-tenth or less the concentration of the competitor.

11. The method of claim 9, wherein the competitor is in about 10-fold excess over a concentration equivalent to its K_{d} for binding to the cognate binding partner.

12. The method of claim 2, wherein the competitor is expressed from an expression vector comprising nucleic acid sequences encoding the competitor that is introduced into the cell population.

13. The method of claim 12, wherein the competitor and the cognate binding partner linked to fragment B of the marker are encoded on one expression vector.

14. The method of claim 13, wherein the competitor and cognate binding partner linked to fragment B are expressed as a dicistronic transcript from a single promoter.

15. The method of claim 14, wherein the promoter is a trp-lac promoter.

16. The method of claim 2, wherein the bacterial cells are gram negative bacteria.

17. The method of claim 2, wherein the marker comprises a signal peptide.

18. A bacterial cell comprising:
an expression vector comprising nucleic acid sequences encoding a member of a library of test binding pair members linked to a fragment A of a marker;
an expression vector comprising nucleic acid sequences encoding a cognate binding partner linked to a fragment B of a marker;
wherein the marker is active when the fragment A and the fragment B are in proximity; and
a competitor that competes with a reference binding pair member for binding to the cognate binding partner and has an affinity for the cognate binding partner at least equal to that of the reference binding pair member.

19. The cell of claim 18, wherein the competitor is the reference binding pair member.

20. The cell of claim 18, wherein the reference binding pair member is an antibody.

21. The cell line of claim 20, wherein the antibody is a single chain antibody.

22. The cell of claim 21, further wherein the test binding pair members linked to fragment A are single chain antibodies.

23. The cell of claim 18, wherein the cognate binding partner linked to fragment B is expressed at a concentration that is limiting, the competitor is expressed in an amount that is in excess over its K_{d} for binding to the cognate binding partner, and the concentration of the test binding pair member linked to fragment A expressed in the cell population is substantially the same as that of the cognate binding partner linked to fragment B.

24. The cell of claim 18, wherein the concentration of the cognate binding partner linked to fragment B is one-tenth or less the concentration of the competitor.

25. The cell of claim 18, wherein the competitor is in about 10-fold excess over its K_{d} for binding to the cognate binding partner.

26. The cell of claim 18, wherein the competitor is expressed from an expression vector comprising nucleic acid sequences encoding the competitor.

27. The cell of claim 26, wherein the competitor and the cognate binding partner linked to fragment B of the marker are encoded on one expression vector.

28. The cell of claim 27, wherein the competitor and cognate binding partner linked to fragment B are expressed as a dicistronic transcript from a single promoter.

29. The cell of claim 28, wherein the promoter is a trp-lac promoter.

30. The cell of claim 18, wherein the cell is a gram negative bacterial cell.

31. The cell of claim 18, wherein the marker comprises a signal peptide.
